(19) United States
(12) Patent Application Publication
(10) Pub. No.: US 2018/0181689 A1
(43) Pub. Date: Jun. 28, 2018

(54) METHODS FOR ESTIMATING A DISINFESTATION RATE OF DISINFESTATION EQUIPMENT

(71) Applicant: Ziel Equipment, Sales and Services, LLC, San Francisco, CA (US)

(72) Inventors: Hon Ming CHUN, Lexington, MA (US); Andrew ROSS, Berkeley, CA (US)

(21) Appl. No.: 15/388,988

(22) Filed: Dec. 22, 2016

(51) Int. Cl. G06F 17/50; C12Q 1/22; A61L 2/22; A23L 3/00

(52) U.S. Cl. CPC G06F 17/5009; C12Q 1/22; A23V 2002/00; A23L 3/00; A61L 2/22

(57) ABSTRACT

Methods for accurately and efficiently estimating a disinfestation rate of disinfestation equipment and making adjustments based on the estimated disinfestation rate to achieve a desired disinfestation rate are provided. In one example, a disinfestation rate may be based on a number of infested products in an untreated sample out of a total number of products in the untreated sample and based on a number of infested products in a treated sample out of a total number of products in the treated sample. The estimated disinfestation rate may displayed, for example, and parameters of the disinfestation equipment may be adjusted in response to the estimated disinfestation rate

### BACKGROUND/SUMMARY

The ability to validate the efficiency of disinfestation equipment is important for ensuring that products being processed by such disinfestation equipment are not contaminated. For example, in the food industry, many food products are processed with disinfestation equipment in order to kill unwanted organisms in the food products. Thus, validating that the disinfestation equipment is effective in killing these unwanted organisms may be desired. Furthermore, food processors may have policies requiring validation of a disinfestation rate for disinfestation equipment in order to use such equipment.

Previously, validation of disinfestation equipment effectiveness has mostly relied upon inoculating a sample with a large number of microbes, processing the inoculated sample with the disinfestation equipment, and then counting a number of microbes that survived in the sample processed by the disinfestation equipment. Additionally, other previous approaches may have included running experiments, such as laboratory experiments, to estimate a disinfestation rate of the disinfestation equipment. In such experiments, all of the products processed by the disinfestation equipment may be inspected for infestation prior to and following the processing in order to estimate a disinfestation rate.

However, the inventors have realized several drawbacks to these above approaches. For example, inoculation of samples with a large number of organisms may not be possible for validating the effectiveness of the disinfestation equipment for killing organisms larger in size, such as insects. In particular, in may not be possible to inoculate a sample with a large enough number of insects to obtain statistically significant results. Furthermore, validating an effectiveness of disinfestation equipment via the above described experimental method that may include inspecting all of the products for infestation may lead to product waste, as inspecting products for infestation may require cutting the products open, thus rendering the products unfit for sale, in some examples.

In such examples where inspecting the products for infestation may render the products unfit for sale, approaches where a large portion of products or all products may need to be inspected for infestation may not be feasible. For example, in scenarios where a disinfestation rate may need to be estimated for disinfestation equipment of a food processing facility, it may not be feasible to inspect a large portion or all of the products that are being processed, especially if inspection renders the food products unsellable. Such approaches may not be feasible due to the economic impacts of rendering a large portion or all of a total number of products unsellable, for example.

To at least partially address these above issues, the inventors have developed methods for estimating a disinfestation rate of disinfestation equipment. In one example, these methods may include determining infestation information and estimating a disinfestation rate based on the determined infestation information. Determining the infestation information may include determining a number of products that are infested in a first sample out of a total number of products in the first sample, where the first sample is untreated by the disinfestation equipment, and determining a number of products that are infested in a second sample out of a total number of products in the second sample, where the second sample has been treated by the disinfestation equipment. In at least one example, these products may be food products, and the disinfestation equipment may be processing the food products with one or more treatments for killing organisms in the food products. It is noted that reference to the disinfestation equipment processing the food products with one or more treatments may also be referred to as the disinfestation equipment treating the products herein. Additionally, an infested product in the first sample and the second sample may be a product that includes any live infestation.

In at least one example, estimating the disinfestation rate based on the determined infestation information may include estimating a survival rate based on the determined infestation information and updating an aggregated survival likelihood function with the estimated survival rate. The disinfestation rate may then be estimated based on the updated aggregated survival likelihood function.

Updating an aggregated survival likelihood function for a survival rate with the estimated survival rate to estimate the disinfestation rate may enable an ongoing estimation of the disinfestation rate, where the disinfestation rate may be estimated over multiple runs. The ongoing estimation of the disinfestation rate may increase the accuracy of the disinfestation rate estimate. Additionally, the ongoing estimation of the disinfestation rate may enable a manufacturer to more accurately and efficiently estimate a disinfestation rate of the disinfestation equipment compared to methods that estimate a disinfestation rate during a single run. Furthermore, via the methods developed by the inventors, sampling rates, that is a number of products in each untreated and treated sample, may be varied from sampling event to sampling event. Thus, the approaches described herein may provide a feasible and economically advantageous manner for estimating a disinfestation rate compared to previous approaches. In particular, the approaches described herein may be advantageous for estimating a disinfestation rate of disinfestation equipment that is processing products that have a low infestation rate. For example, a low infestation rate of the products may be an infestation rate of less than approximately 10%. It is noted that disinfestation may refer to eradication of vermin in at least one example. Examples of vermin may include arthropods such as crustaceans (e.g., crabs, lobsters, crayfish, shrimp), arachnids (e.g., spiders, scorpions, ticks, mites), and insects (e.g., beetles, bugs, earwigs, ants, bees, termites, butterflies, moths, crickets, roaches, fleas, flies, mosquitoes, lice). Additionally, other examples of vermin may include rodents such as mice. However, vermin may include any objectionable small animals. In some examples, the disinfestation rate estimated herein may only be for estimating a disinfestation rate of vermin (i.e., small animals as described above). However, additionally or alternatively, the disinfestation rate may be estimated for microbes, such as bacteria.

### BRIEF DESCRIPTION OF THE FIGURES

FIG. 1 shows a block diagram of an example environment according to at least one embodiment of the present disclosure.

FIG. 2 shows a block diagram of an example disinfestation estimating device.

FIG. 3 shows a flow chart of an example method for estimating a disinfestation rate.

FIG. 4 shows a flow chart of an example method for calculating the disinfestation rate.

FIG. 5 shows a graph of an example aggregated likelihood function for a survival rate.

FIG. 6 shows a graph of an example pass-fail curve plotted against survival rate estimate.

### DETAILED DESCRIPTION

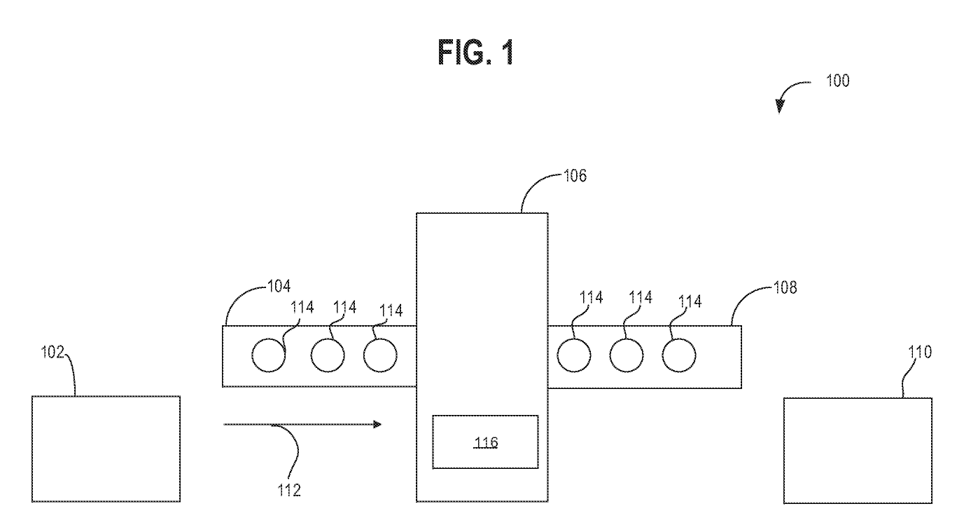

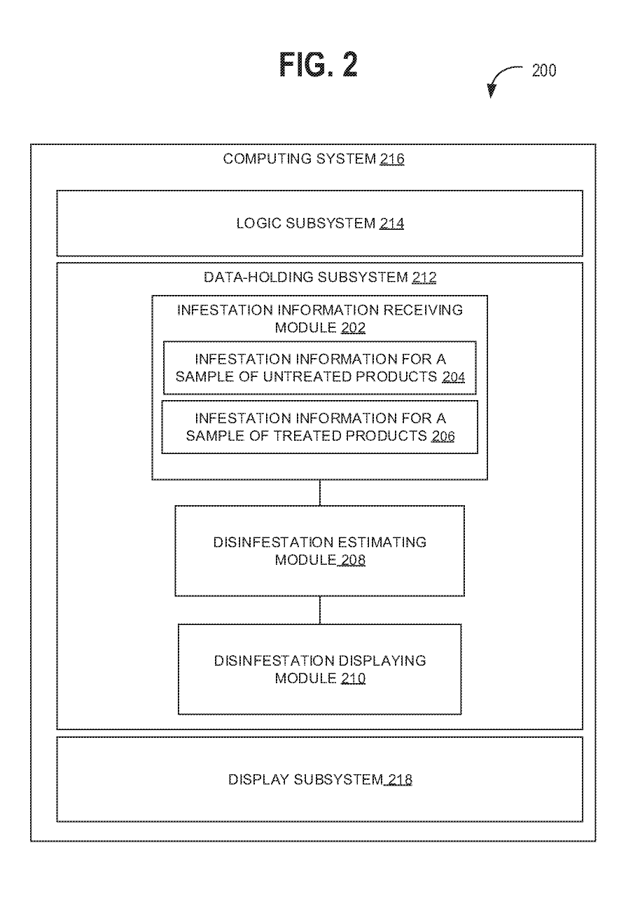

Methods for accurate and efficient estimation of a disinfestation rate for disinfestation equipment are provided. These methods may be carried out in a food processing environment, such as the food processing environment described in relation to FIG. 1, and these methods may be carried out by a disinfestation estimation device, as described in FIG. 2. Based on the estimated disinfestation rate, mitigating action may be taken. For example, operating parameters of the disinfestation equipment may be adjusted or the disinfestation equipment may be inspected for malfunctions.

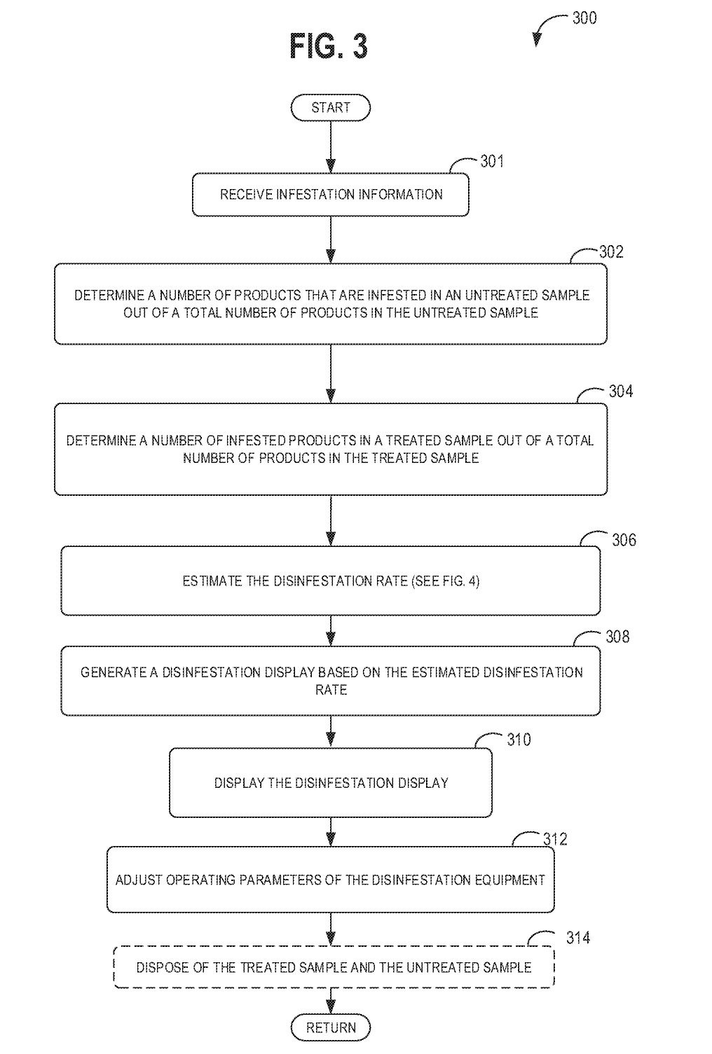

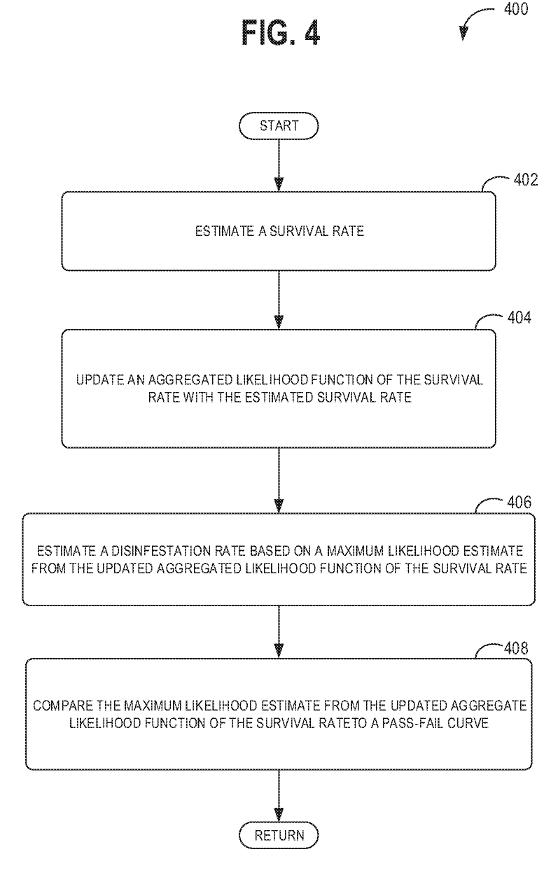

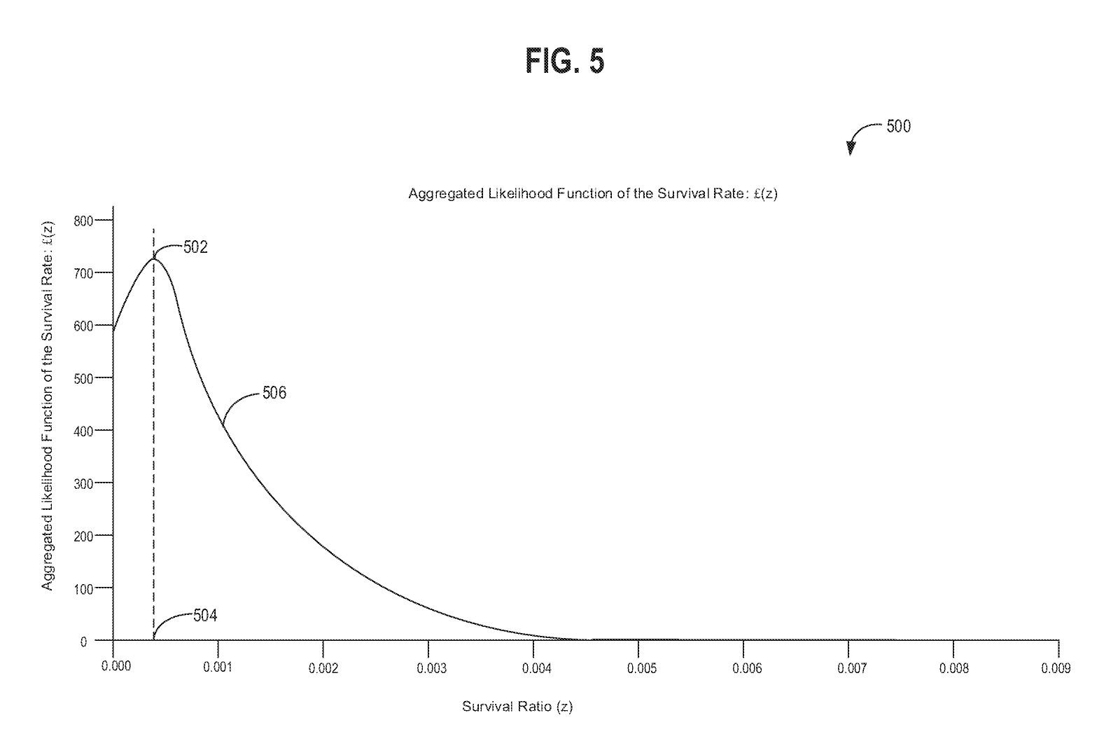

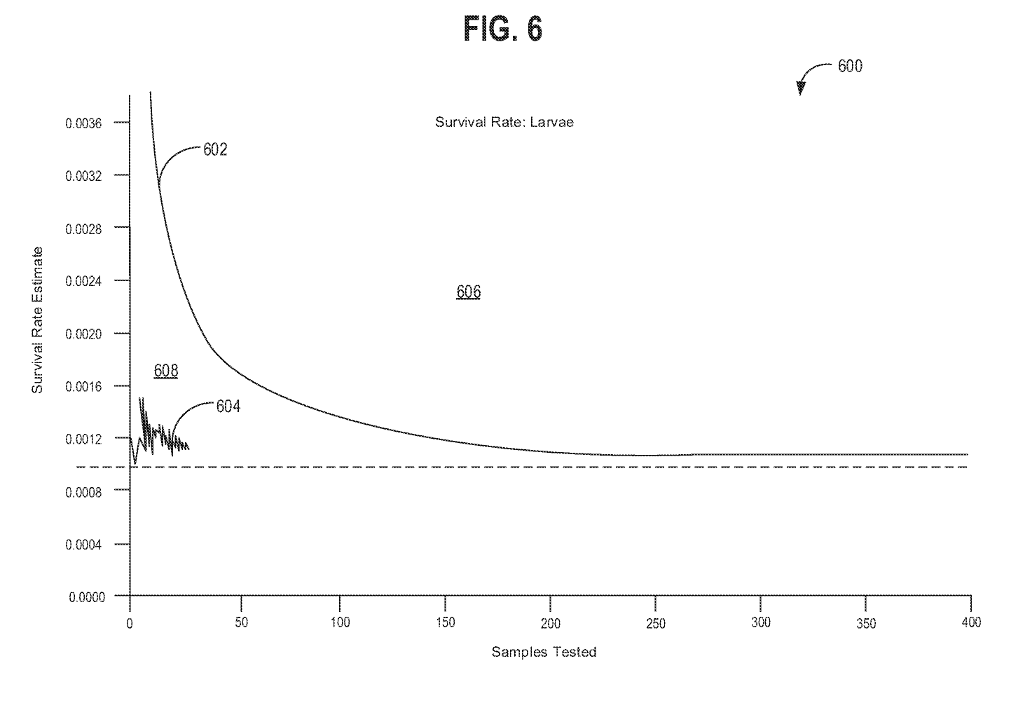

Methods for estimating the disinfestation rate of disinfestation equipment, such as shown at FIG. 3, may include determining infestation information of an untreated sample and in a treated sample. The disinfestation rate may then be determined based upon this infestation information of the treated and the untreated samples to estimate a disinfestation rate. In some examples, the estimated disinfestation rate and other disinfestation information may be displayed. Furthermore, operating parameters of the disinfestation equipment may be adjusted. In at least one example, the disinfestation rate may be estimated via calculations as described at FIG. 4. In addition to the calculations described at FIG. 4, calculating the disinfestation rate estimate may include comparing various graphs, such as the graphs at FIGS. 5 and 6, to determine the estimated disinfestation rate and to confirm whether the disinfestation rate estimate passes or fails a pass-fail curve for reaching a desired disinfestation estimate.

Turning to FIG. 1, FIG. 1 shows an example environment 100 in which the provided method may be carried out. Environment 100 may be a food processing environment including pallets 102 containing food products 114 that are untreated, disinfestation equipment 106, and a disinfestation estimating device 110. In at least one example, the disinfestation equipment 106 may be equipment that processes the food products 114 with one or more treatments in order to kill unwanted organisms, such as insects. For example, in one embodiment the disinfestation equipment 106 may be used to treat food products such as dates to kill live infestations of insects within the dates. It is noted that reference to a product herein, such as a food product, may refer to a single item or single unit, and reference to a product herein is not reference to a commodity. For example, a product may be a single fruit, such as a single date. The term products thus refers to multiple items or units. For example, products may refer to multiple food products such as multiple dates.

In at least one example, the food products 114 may be conveyed through the disinfestation equipment 106 via a conveying device 104, 108. The conveying device is comprised of an upstream portion 104 and a downstream portion 108 relative to the disinfestation equipment 106. For example, the upstream portion 104 of the conveying device may be a portion of the conveying device that is upstream the disinfestation equipment 106, and the downstream portion 108 of the conveying device 108 may be a portion of the conveying device that is downstream the disinfestation equipment 106. Additionally or alternatively, the food products may be processed in a batch manner, where food products may be manually placed in a chamber of the disinfestation equipment 106, the disinfestation equipment 106 may process the food products with one or more treatments, and then the treated food products may be manually removed from the chamber of the disinfestation equipment 106. In embodiments where the food products may be processed in a batch manner, the disinfestation equipment 106 may not include a conveying device 104, 108. However, embodiments where the food products are processed in a batch manner, the disinfestation equipment 106 may still include a conveying device in at least one example.

The upstream portion of the conveying device 104 may convey food products 114 that are untreated food products, where the untreated food products are food products that have not been treated by the disinfestation equipment 106. The conveying device 104, 108 may carry the food products 114 in a direction 112 from an upstream portion 104 of the conveying device relative to the disinfestation equipment, through the disinfestation equipment 106, and to a downstream portion 108 of the conveying device relative to the disinfestation equipment. Thus, in one example, untreated food products 114 may be transported from upstream the disinfestation equipment 106 and through the disinfestation equipment 106. While the untreated food products 114 are within the disinfestation equipment 106, the disinfestation equipment 106 may process the untreated food products with one or more treatments. For example, the disinfestation equipment 106 may process the untreated food products with any one or combination of treatments that may include microwave or other forms of radiofrequency processing, thermal processing, Pascalization, oxygen depletion, radiation, chemical application, water activity reduction, and pH adjustments. By processing the untreated food products with any one or combination of treatments, the disinfestation equipment 106 may kill unwanted organisms in the food products, such as insects.

The specific treatments performed by the disinfestation equipment on the products may be determined based on the required living conditions of an organism or plurality of organisms that are targeted to be killed in the food products. For example, the specific treatments that the disinfestation equipment may perform on the products may be treatments to expose the products to conditions which the organism or organisms desired to be killed may not be able to survive.

In some examples, these organisms may be organisms that are large enough in size to be detected by visually inspecting the food products to be treated by the disinfestation equipment without a visual aid such as a microscope. For example, these larger organisms may be insects or other vermin.

In one example, thermal processing treatments, such as microwave or other radiofrequency treatments, performed by the disinfestation equipment may include one or both of increasing a temperature of the food products to be greater than a first threshold temperature and decreasing a temperature of the food products to be less than a second threshold temperature, the first and second threshold temperatures based on temperature tolerances for an organism or plurality of organisms that are desired to be killed via the disinfestation equipment. For example, the disinfestation equipment may increase an internal temperature of the food products to temperatures greater than the first threshold temperature, where the first threshold temperature is a temperature greater than a temperature that may be tolerated by the organism or organisms that are desired to be eradicated by the disinfestation equipment.

The first temperature threshold may also be based on a temperature that would degrade the products, such as food products, being treated. For example, the first temperature threshold may be a temperature that is high enough to kill the organism or organisms that are desired to be killed via the disinfestation equipment while still being a low enough temperature to avoid degradation of the products being treated.

In some examples, the disinfestation equipment, such as microwave or other radiofrequency based disinfestation equipment, may increase an internal temperature of the food products to a temperature less than the second threshold temperature, where the second threshold temperature is a temperature lower than a livable temperature for the organism or organisms desired to be eradicated from the products by the disinfestation equipment.

Additionally or alternatively, the disinfestation equipment may perform a pressure treatment on the food products, also referred to as Pascalization, where the disinfestation equipment may increase a pressure within the food products to pressures greater than a threshold pressure. In at least one example, the pressure threshold may be a pressure that is greater than a pressure for organism or organisms that are desired to be eradicated to survive. Additionally, the pressure threshold may be a pressure that is low enough to avoid degradation of the food products being treated.

The disinfestation equipment may additionally or alternatively perform an oxygen deprivation treatment on the food products. In such examples where the food products may undergo an oxygen deprivation treatment, the products may be held in a chamber, and an atmosphere within the chamber may be modified to reduce an oxygen level in the chamber below a threshold oxygen level. In some examples, the threshold oxygen level may be an oxygen level that is less than a required oxygen level for the organism or organism desired to be eradicated by the disinfestation equipment. The oxygen level may be reduced below the threshold oxygen level via displacing oxygen within the chamber with carbon dioxide (CO₂) or nitrogen gas (N₂), for example.

Another example treatment that the disinfestation equipment may perform on the products may include a radiation treatment. For example, the disinfestation equipment may expose the food products to ionizing radiation in order to eradicate the presence of unwanted organisms. In some examples, the food products may be irradiated via beta particles or gamma rays.

Further still, another treatment that the disinfestation equipment may perform on the food products may additionally or alternatively include chemical application. For example, glutaraldehyde may be applied to the food products to kill unwanted organisms, such as insects.

The disinfestation equipment may additionally or alternatively perform a water activity reduction treatment to the food products to reduce the water activity below a threshold water activity. The threshold water activity may be a water activity that may prevent or inhibit growth of unwanted organisms, in at least one example. In some examples, a water activity of the food products may be reduced by salting the food products or dehydrating the food products. Furthermore, the disinfestation equipment may additionally or alternatively perform a pH treatment on the food products to decrease the pH to be less than a pH threshold. The pH threshold may be a pH that prevents or inhibits the growth of unwanted organisms. In some examples, the pH may be reduced by adding acid to the food products being processed.

Following treatment of the untreated food products via the disinfestation equipment 106, the food products may be conveyed to a downstream portion 108 of the conveying device relative to the disinfestation equipment. After food products 114 have been conveyed through the disinfestation equipment 106 and processed by the disinfestation equipment 106 with any one or combination of treatments, these food products 114 may be referred to as treated food products. The treated food products may be conveyed to a downstream portion 108 of the conveying device. Following conveying the treated food products to a downstream portion 108 of the conveying device, these treated food products may be packaged to be sold or these food products may be sampled as a part of a treated sample to estimate a disinfestation rate of the disinfestation equipment.

It may be desirable to estimate a disinfestation rate of the disinfestation equipment 106 in order to validate that unwanted organisms, such as insects, are being eradicated from the food products 114 being treated by the disinfestation equipment 106.

In order to estimate the disinfestation rate, a disinfestation estimating device 110 may be used. When estimating the disinfestation rate of the disinfestation equipment 106, a sample of untreated food products and a sample of treated food products may be inspected to determine infestation information. In some examples, the sample of untreated food products may be taken directly from the pallet 102, prior to any processing of the food products. However, in other examples, the sample of the untreated food products may be taken from an upstream portion 104 of the conveying device relative to the disinfestation equipment 106, prior to the food products being treated via the disinfestation equipment. Additionally, a sample of treated food products that may be taken, where the sample of treated food products may been treated via the disinfestation equipment 106.

In at least one example, in order to gather an untreated sample and a treated sample of food products, a portion of food products out of a total number of food products may be treated with the disinfestation equipment and a remaining portion of food products out the total number of food products that are not treated may be left untreated by the disinfestation equipment. Then, infestation information for the food products untreated by the disinfestation equipment may be determined and infestation information for the food products treated with the disinfestation equipment may be determined.

For example, out of the products left untreated by the disinfestation equipment, a portion of these untreated products may be inspected. In other examples, however, all of the products left untreated by the disinfestation equipment may be inspected. These untreated products that are inspected may be referred to as an untreated sample.

Similarly, out of the products treated by the infestation equipment, a portion of these treated products may be inspected. In other examples, however, all of the products treated by the disinfestation equipment may be treated in at least one example. These treated food products that are inspected may be referred to as a treated sample.

When inspecting the untreated and the treated, infestation information for the untreated and treated samples may be determined. This infestation information may be provided to a disinfestation estimating device, and then a disinfestation rate of the disinfestation equipment may be determined based on the determined infestation information for the food products untreated by the disinfestation equipment and the food products treated with the disinfestation equipment.

In some examples, the food products for the untreated sample and the treated sample may be taken at the same time. Alternatively, the food products for the treated sample may be taken prior to taking the food products for the untreated sample. Thus, in such examples, the food products for the untreated sample may be different from the food products for the treated sample.

However, in other examples, the food products for the untreated sample and the food products for the treated sample may be the same food products. In such examples where the food products are the same for the treated and the untreated samples, the untreated sample of food products may first be inspected to determine infestation information for the untreated sample, and then this inspected untreated sample may be processed by the disinfestation equipment 106 with any one or combination of treatments. After the untreated food products that have been inspected once prior to processing have been processed by the disinfestation equipment, these same food products, which are now treated food products, may be inspected a second time, following treatment, to determine infestation information for the treated sample.

In at least one example, the infestation information gathered for the above described untreated and treated samples may include a number of infested food products and a total number of products in each sample. For example, each food product taken for an untreated sample may be inspected to determine whether or not any of the untreated food products contain a live infestation. Each food product that may be determined to contain any live infestation may be determined to be an infested food product, and a total number of the food products inspected in this untreated sample may be counted to determine the total number of food products for the untreated sample. Similarly, for the treated sample, each food product taken for the treated sample may be inspected to determine whether or not any of the treated food products contain a live infestation, and a total number of the food products in this treated sample may be counted to determine a total number of food products for the treated sample.

In at least one example, sampling a product for either or both of an untreated and a treated sample may include taking a representative sample. For example, a representative untreated sample may be taken by collecting and mixing a total number of possible untreated food products that may be used for an untreated sample. In some embodiments, the untreated food products may be taken from multiple locations throughout a container of untreated food products that are to be processed, such as a pallet received by a food processing plant. All of these collected untreated food products may then be mixed. This mixing of food products that are taken from multiple locations throughout the container may reduce a bias that could be introduced if food products from only a portion of the container were taken. Once the untreated food products are mixed, a portion of these untreated food products may be used for the untreated sample. Thus, the resulting untreated sample may be a representative sample of the entire container of untreated food products from which they were taken.

Similarly, taking a representative treated sample may include collecting and mixing a total number possible treated food products that may be used for a treated sample. For example treated food products throughout a sample of treated food products may be collected and mixed in a single container. In some examples all of the food products that are being treated may be collected and mixed. However, in other examples, only a portion of the food products that are being treated may be collected and mixed.

In embodiments where food may be treated in a batch process, the food products may be taken from multiple locations throughout a container that may hold the food products during the batch processing of the food products. In embodiments where food products may be moved through the disinfestation equipment in a continuous manner, such as via conveyer belts, collecting a representative treated sample may include collecting treated food products at different times throughout a disinfestation run, as the treated food products are conveyed downstream of the disinfestation equipment. Once treated food products throughout a sample of the food products are collected, the treated food products may then be mixed in a single container, and a portion of these mixed treated food products may be selected for a treated food sample. In some examples, inspecting the food products may include scanning the food products via sensors and sending an output from these sensors to the disinfestation estimating device 110. For example, sensors may be disposed upstream and downstream of the disinfestation equipment 106, and these sensors may detect and convey the above discussed information to the disinfestation estimating device 110.

However, in other embodiments, the food products may be manually inspected. For example, the food products may be visually inspected. In at least one example, visually inspecting the food products may include inspecting the food products without a microscope. In examples where the food products may be manually inspected, the food products may be cut open in order to determine whether or not any of the food products contain a live infestation. Food products may be determined to contain a live infestation if they contain at least one live insect, for example.

Once infestation information for the treated sample and the untreated sample have been determined, the infestation information may then be received by the disinfestation estimating device, and the disinfestation estimating device may then estimate a disinfestation rate. For example, the disinfestation estimating device may receive the infestation information via a user input or via communication with a control unit 116 of the disinfestation equipment 106. More example details regarding methods for estimating the disinfestation rate may be described in relation to FIGS. 2-6.

Based on an output from the disinfestation estimating device 110, adjustments may be made to operating parameters of the disinfestation equipment 106. For example, the disinfestation estimating device 110 output may indicate an estimated disinfestation rate. Additionally or alternatively, the disinfestation estimating device 110 output may indicate any one or combination of calculations and models generated while determining an estimate for the disinfestation rate of the disinfestation equipment. Specific calculations and models that may be generated are discussed in more detail below.

Adjustments may be made to operating parameters of the disinfestation equipment 106 responsive to the disinfestation estimating device estimating that the disinfestation rate is less than a desired disinfestation rate, for example, and these parameter adjustments to the disinfestation equipment 106 may be in order to achieve the desired disinfestation rate. In at least one example, the disinfestation estimating device 110 may automatically adjust operating parameters of the disinfestation equipment 106, or generate indications of how parameters of the equipment should be adjusted (said indications displayed on a display, for example). For example, the disinfestation estimating device 110 may automatically adjust parameters of the disinfestation equipment 106 via communication with a control unit 116 of the disinfestation device. In examples where operating parameters of the disinfestation equipment may be adjusted automatically via communication between the control unit 116 of the disinfestation equipment 106 and the disinfestation estimating device 110, the disinfestation estimating device 110 may communicate with the control unit 116 of the disinfestation device, and the control device 116 may actuate actuators responsive to communication with the disinfestation device to adjust the parameters of the disinfestation equipment. However, in other examples, these operating parameters may be adjusted manually via a user input to the control unit 116 of the disinfestation device in response to the disinfestation information that is displayed or in response to an indications that are generated and displayed for how operating parameters to the disinfestation equipment should be adjusted.

Additionally or alternatively, the disinfestation estimating device 110 may provide a display showing disinfestation information. For example, the disinfestation estimating device may provide a display showing any one or combination of the disinfestation information as discussed in further detail below.

In examples where the disinfestation estimating device 110 may provide a display showing disinfestation information, adjustments to operating parameters may be manually made to the disinfestation equipment 106 responsive to the displayed disinfestation information. For example, in response to the disinfestation information being displayed, a user may manually adjust operating parameters of the disinfestation equipment 106 via input to a user interface of the control unit 116 of the disinfestation equipment 106, or via input to a device communicatively coupled to the control unit 116.

Such operating parameter adjustments may include any one or more of a combination of adjustments to a processing time of the disinfestation equipment 106 for treating the food products 114, and adjustments to various operating thresholds such as temperature, pressure, oxygen levels, radiation, chemical levels, water activity, and pH thresholds.

For example, a processing time may be increased in order to ensure that products being treated with the disinfestation equipment reach threshold conditions (e.g., temperature, pressure, radiation, chemical, and atmospheric) for eradicating unwanted organisms, such as insects. In at least one example, these parameters of the disinfestation equipment may be adjusted automatically in response to determining that the disinfestation rate is less than the desired disinfestation rate. In examples where the parameters of the disinfestation equipment may be adjusted automatically responsive to estimating the disinfestation rate, it is noted that disinfestation may or may not be displayed.

Additionally, in examples where the disinfestation estimating device 110 may be communicatively linked with the control unit 116 of the disinfestation equipment 110, the parameters of the disinfestation equipment 106 may be adjusted via input to a disinfestation estimating device, and the disinfestation estimating device may then communicate with the control unit 116 to make adjustments to the operating parameters of the disinfestation equipment. For example, the input to the disinfestation estimating device may be a user input received via a user interface, in at least one example.

Adjustments to operating parameters of the disinfestation equipment 106 may be carried out by adjusting actuators of various components of the disinfestation equipment 106 that may change such parameters. In at least one example, these actuators may be controlled via control unit 116 of the disinfestation equipment 106.

For example, in at least one embodiment, a processing time may be an operating parameter that is adjusted responsive to estimating the disinfestation rate via the disinfestation device 110. In some examples, adjusting a processing time may include actuating motors of a conveyer belt of the disinfestation equipment to increase or decrease a speed of the conveyer belt, thus increasing or decreasing a processing time of product by the disinfestation equipment. For example, actuating motors to decrease a speed of the conveyer belt may increase the processing time, and increasing the processing time for disinfestation equipment that thermally treats food products may increase a disinfestation rate, in some examples. In particular, increasing the processing time may enable a temperature of the food products to increase to greater than a threshold temperature or to decrease below a threshold temperature during thermal processing for heating and cooling treatments, respectively. Therefore, in examples where the estimated disinfestation rate may be less than a desired disinfestation rate for disinfestation equipment that thermally treats the food products, the processing time may be increased to increase the disinfestation rate to be greater than or equal to the desired disinfestation rate.

In examples where an atmosphere may be modified to kill unwanted organisms, the processing time may be adjusted by increasing a length of time or decreasing a length of time that products are held within an oxygen depleted environment of the disinfestation equipment. For example, actuators for opening and closing a chamber of the disinfestation equipment may be held closed for a longer or shorter period of time. Holding the chamber closed for a longer period of time may increase a processing time, and holding the chamber closed for a shorter period of time may decrease the processing time. Increasing a processing time for holding the food products in an oxygen depleted environment may increase a disinfestation rate. Therefore, in examples where the estimated disinfestation rate may be less than a desired disinfestation rate, then the processing time may be increased to increase the disinfestation rate to be greater than or equal to the desired disinfestation rate.

Additionally or alternatively, other example adjustments may include increasing a temperature by actuating a heating element actuator to increase an output of the heating element. Increasing the output of the heating element may help to ensure that food products are heated to greater than the first temperature threshold for killing unwanted organisms. Thus, if the estimated disinfestation rate is less than a desired disinfestation rate then the output of the heating element may be increased in order to increase the estimated disinfestation rate to be greater than or equal to the desired threshold.

Adjustments to decrease a temperature may be achieved via actuating a cooling element actuator to increase a flow rate of cooling fluid, for example. Increasing a flow rate of cooling fluid may help to ensure that a temperature of the food products are decreased below the second temperature threshold temperature for killing unwanted organisms. Thus, if the estimated disinfestation rate is less than a desired disinfestation rate then the flow rate of cooling fluid may be increased in order to increase the estimated disinfestation rate to be greater than or equal to the desired threshold.

Furthermore, adjusting other disinfestation equipment parameters may include adjusting other actuators to adjust such parameters, such as motors, pumps, electrical pulsing, etc. These parameters may be adjusted responsive to an estimation of the disinfestation rate that is less than the desired disinfestation rate, for example, and the adjustments made to the parameters of the disinfestation equipment 106 may be made in order to increase the disinfestation rate to greater than or equal to the desired disinfestation rate.

Furthermore, the disinfestation equipment may be inspected responsive to displayed disinfestation information indicating that the estimated disinfestation rate is less than a desired disinfestation rate. For example, any one or combination of motors, pumps, heating elements, cooling elements, sealing elements, chemical levels in storage tanks, and other components of the disinfestation equipment may be inspected to determine if there is a malfunction in the machinery causing the estimated disinfestation rate to fall below the desired disinfestation rate.

In at least one example, this disinfestation equipment may be inspected automatically by running one or a plurality of diagnostic tests via the control unit 116. However, in other examples, the disinfestation equipment may be inspected by a user. For example, a user may visually inspect the disinfestation equipment to determine if there are any components of the disinfestation equipment that may be malfunctioning.

Turning now to FIG. 2, FIG. 2 shows a disinfestation estimating device 200 for estimating a disinfestation rate of disinfestation equipment. The disinfestation estimating device 200, which may correspond with the disinfestation estimating device 110 described in relation to FIG. 1, may be a computing system 216 including a logic subsystem 214 and a data-holding subsystem 212.

Computing system 216 schematically shows a non-limiting computing system that may perform one or more of the methods and processes described herein. For example, the instructions stored in data-holding subsystem 212 may be instructions executable by the logic subsystem 214 to implement the herein described methods and processes disclosed at FIGS. 3-6. It is to be understood that virtually any computer architecture may be used for a computing device without departing from the scope of this disclosure. For example, the architecture shown in FIG. 2 for the data-holding subsystem 212 may differ, so that one or more of the modules 202-210 may be configured for performing more or fewer tasks. In different embodiments, computing system 216 may take the form of a mainframe computer, server computer, desktop computer, laptop computer, tablet computer, home entertainment computer, network computing device, mobile computing device, mobile communication device, gaming device, etc.

Computing system 216 includes a logic subsystem 214 and a data-holding subsystem 212. Computing system 216 may optionally include other components not shown in FIG. 2. For example, computing system 216 may also optionally include user input devices such as keyboards, mice, game controllers, cameras, microphones, and/or touch screens.

Logic subsystem 214 may include one or more physical devices configured to execute one or more instructions. For example, logic subsystem 214 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

Logic subsystem 214 may include one or more processors that are configured to execute software instructions. Additionally or alternatively, the logic subsystem 214 may include one or more hardware or firmware logic machines configured to execute hardware or firmware instructions. Processors of the logic subsystem 214 may be single core or multi-core, and the programs executed thereon may be configured for parallel or distributed processing. The logic subsystem 214 may optionally include individual components that are distributed throughout two or more devices, which may be remotely located and/or configured for coordinated processing. One or more aspects of the logic subsystem 214 may be virtualized and executed by remotely accessible networked computing devices configured in a cloud computing configuration.

Data-holding subsystem 212 may include one or more physical, non-transitory devices configured to hold data and/or instructions executable by the logic subsystem 214 to implement the herein described methods and processes. When such methods and processes are implemented, the state of data-holding subsystem may be transformed (for example, to hold different data).

Data-holding subsystem 212 may include removable media and/or built-in devices. Data-holding subsystem 212 may include optical memory (for example, CD, DVD, HD-DVD, Blu-Ray Disc, etc.), and/or magnetic memory devices (for example, hard disk drive, floppy disk drive, tape drive, MRAM, etc.), and the like. Data-holding subsystem 212 may include devices with one or more of the following characteristics: volatile, nonvolatile, dynamic, static, read/write, read-only, random access, sequential access, location addressable, file addressable, and content addressable. In some embodiments, logic subsystem 212 and data-holding subsystem 212 may be integrated into one or more common devices, such as an application specific integrated circuit or a system on a chip.

It is to be appreciated that data-holding subsystem 212 includes one or more physical, non-transitory devices. In contrast, in some embodiments aspects of the instructions described herein may be propagated in a transitory fashion by a pure signal (for example, an electromagnetic signal, an optical signal, etc.) that is not held by a physical device for at least a finite duration. Furthermore, data and/or other forms of information pertaining to the present disclosure may be propagated by a pure signal.

When included, display subsystem 218 may be used to present a visual representation of data held by data-holding subsystem 212. As the herein described methods and processes change the data held by the data-holding subsystem 212, and thus transform the state of the data-holding subsystem 212, the state of display subsystem 218 may likewise be transformed to visually represent changes in the underlying data. Display subsystem 218 may include one or more display devices utilizing virtually any type of technology. Such display devices may be combined with logic subsystem 214 and/or data-holding subsystem 212 in a shared enclosure, or such display devices may be peripheral display devices.

It is noted that the control unit 116 of the disinfestation equipment described in FIG. 1 may also be a computing system. Thus, the control unit 116 may also include at least a portion of the features discussed in relation to the disinfestation estimating device 200. For example, the control unit 116 may include a logic subsystem and a data-holding subsystem, where the data-holding subsystem may hold instructions executable by the logic subsystem for carrying out the disinfestation treatment processes. For example, the data-holding subsystem of the control unit 116 of the disinfestation equipment 106 may include instructions executable by the logic subsystem to control actuators of the disinfestation equipment 106 based on receiving an input. Additionally, the data-holding subsystem 212 of the control unit 116 may hold instructions executable by the logic subsystem 214 for controlling the actuators of the disinfestation equipment 106 responsive to an input. In at least one example, the input may be an input to a user interface of the control unit 116 or the input may be from a device, such as a disinfestation estimating device 200, that is communicatively linked with the disinfestation equipment 106. The input may be a request to adjust operating parameters of the disinfestation equipment 106, in at least one example. Furthermore, the request to adjust the operating parameters of the disinfestation equipment 106 may be made automatically in response to an output of the disinfestation estimating device 200, or in response to a manual input. For example, the manual input may include an input to a user interface of the control unit 116 or an input to a device communicatively linked to the control unit 116, such as a disinfestation estimating device 200.

Turning back now to the disinfestation estimating device 200, in at least one example, the data-holding subsystem 212 may include an infestation information receiving module 202, a disinfestation estimating module 208, and a disinfestation displaying module 210. These modules stored within the data-holding subsystem 212 of the computing system 216 may be instructions executable by the logic subsystem 214 to implement the herein described methods and processes.

In one embodiment, the infestation information receiving module 202 may be configured to receive infestation information and output this infestation information to the disinfestation estimating module 208. Then, the disinfestation estimating module 208 may estimate a disinfestation rate based on the infestation information received from the infestation information receiving module 202. The infestation information may be received by the infestation information receiving module 202 via user input, in at least one example. In some examples, the user input may be received via a user interface of the computing system, where the user interface may include any one or combination of a graphical user interface, a microphone for receiving auditory instructions, a mouse, a keyboard, a touch screen, and an optical recognition device.

Once the disinfestation estimating module 208 may estimate a disinfestation rate based on the infestation information received by the infestation information receiving module 202, the disinfestation displaying module 210 may provide a display of the estimated disinfestation rate via the display subsystem 218. Additionally or alternatively, the disinfestation displaying module 210 may also display other information determined by the disinfestation estimating module 208. For example, the disinfestation estimating module 208 may perform various calculations and generate various models while estimating a disinfestation rate, and the disinfestation displaying module 210 may display any one or combination of these calculations and models. Furthermore, the disinfestation estimating module 208 perform a pass-fail calculation when estimating the disinfestation rate, and the results of this pass-fail calculation may additionally or alternatively be displayed via the disinfestation displaying module 210.

In some examples, the disinfestation estimating device 200 may be separate from the disinfestation equipment for which the disinfestation estimating device 100 is estimating a disinfestation rate. However, in at least one example, the disinfestation estimating device 200 may be physically or communicatively integrated with the disinfestation equipment for which the disinfestation estimating device 200 is estimating a disinfestation rate. In such examples where the disinfestation estimating device 200 may be communicatively integrated with the disinfestation equipment, the disinfestation estimating device 200 may be part of a control unit of the disinfestation equipment or the disinfestation estimating device 200 may be in direct communication with the disinfestation equipment. For example, the disinfestation estimating device 200 may be in communication with the disinfestation equipment via a wireless connection, or the disinfestation estimating device 200 may be in communication with the disinfestation equipment via a wired connection.

The user interface may be a graphical user interface (GUI) that may include input fields for receiving the user input. For example, the user interface may include input fields for receiving infestation information. In some embodiments, the user interface may include input fields for receiving one or more of a number of untreated products that were sampled, a number untreated products that had a live infestation, a number of treated products that were sampled, and a number of treated products that had a live infestation. Additionally, the user interface may include input fields for additional information regarding a sampling event, where a sampling event is an event where infestation information is determined for both a treated sample and an untreated sample. In some examples, the fields may include any one or combination of fields for a date the products were treated, a time the products were sampled, an ID number of the sample, and a field for any additional notes. These additional fields may help to track information about the products being sampled, in addition to infestation information. In at least one example, these input fields may be used to update or correct previously entered data.

Furthermore, in one example, the infestation information receiving module 202 may receive infestation information via communication with a device that may be communicatively linked to the disinfestation estimating device 200. In one example, the infestation information receiving module may receive infestation information via communication with a device that may be wirelessly linked to the disinfestation estimating device 200. For example, the infestation information receiving module 202 may receive infestation information that is transmitted from a device wirelessly linked with the disinfestation device 200. In some examples, the device wirelessly linked with the disinfestation device 200 may be a device such as a mobile communication device.

The infestation information received by the infestation information receiving module 202 may include infestation information for a sample of untreated products 204 and infestation information for a sample of treated products 206. The infestation information may be determined as described in FIG. 1, for example. It is noted that reference to products herein may refer to food products, in at least one example.

The sample of untreated products may be a sample of products that have not been treated by disinfestation equipment, and the sample of treated products may be items that have been treated by the disinfestation equipment. The infestation information for the sample of untreated products 204 received by the infestation information receiving module 202 may include a number of products that are infested in the untreated sample and a total number of products in the untreated sample. A product may be determined to be infested if the product contains any live infestation. For example, a product may be determined to be infested if the product contains at least one live insect. The number of infested products may be determined via visual examination without the aid of a microscope. In other words, the infestation that may be determined may be an infestation of organisms large enough in size to see without the aid of a microscope.

The total number of products in the untreated sample may be a total number of products that are inspected in the untreated sample. For example, if 200 untreated products are inspected and 15 of the 200 untreated products are determined to contain a live infestation, then the number of infested products in this untreated sample would be 15, and the total number of untreated products in this untreated sample would be 200.

The infestation information for the sample of treated products 206 received by the infestation information receiving module 202 may include a number of products that are infested in the treated sample and a total number of products in the treated sample. As discussed above, a product may be determined to be infested if the product contains any live infestation. For example, the product may be determined to be infested if the product contains at least one live insect. In at least one example, the presence of an infestation in a product may be determined via visual examination, where the visual examination does not include the aid of a microscope. The total number of products in the treated sample may be a total number of products that are inspected in the treated sample.

In at least one example, the treated products that are sampled may be different than the items untreated items that are sampled. However, in some examples, the untreated products that are sampled and the treated products that are sampled may be the same products. The total number of untreated products and the total number of treated products sampled for 204 and 206 may each vary from sampling event to sampling event, where a sampling event is an event that includes sampling of untreated and treated products to determine infestation information. Additionally, the total number of products for the untreated sample may be different from the total number of products for the treated sample. The ability to vary the number of products sampled may be advantageous by providing the ability to take fewer or more products per sample depending on processing needs. For example, if 200 untreated products are sampled but a large percentage of the treated products were damaged due to treatments administered via the disinfestation equipment, then a relatively small number of products for the treated sample may be taken compared to the treated sample.

In one illustrative example for determining infestation information for a sample of untreated products, if 200 treated products are inspected and 1 of the 200 treated products contain a live infestation, then the treated sample would be determined to have 1 infested product and the total number of treated products in this treated sample would be 200. The total number of untreated products sampled may vary from determination of infestation information to determination of infestation information.

By only having to determine if a product contains any live infestation, the advantage of simplified determination of infestation information may be achieved. Previous approaches for determining infestation information to estimate a disinfestation rate of disinfestation equipment may include determining a total number of live organisms in an infested product, as opposed to only having to determine whether or not there is any live infestation at all. Having to determine the total number of live organisms may be time consuming, as this determination may require a more thorough examination of each product. Additionally, having to determine the total number of live organisms may lead to errors due to having to count the number of live organisms and having to record such information regarding the number of live organisms. However, the methods disclosed herein for estimating the disinfestation rate, which is discussed in more detail below, is constructed in order to not need information regarding a specific number of live organisms in a product. Therefore, even though fewer infestation information details may be required when determining infestation information via the disclosed methods, the resulting disinfestation estimate may still be accurate. In contrast, approaches for estimating a disinfestation rate that may require counting each live organism in a sample may result in disinfestation estimates less accurate than the disinfestation estimates resulting from the approach disclosed herein.

Following receiving infestation information for the untreated and the treated samples, the infestation information receiving module 202 may transmit the received infestation information for the untreated sample and the treated sample to the disinfestation estimating module 208. The disinfestation estimating module 208 may receive one or both of infestation information for a sample of untreated products 204 and infestation information for a sample of treated products 206. The disinfestation estimating module 208 may then estimate a disinfestation rate based on the determined infestation information that is received, which is described in more detail at FIG. 4.

Following the disinfestation estimating module 208 estimating a disinfestation rate based on the determined infestation information, the disinfestation estimating module 208 may communicate with the disinfestation displaying module, so that the disinfestation displaying module 210 may generate and then display disinfestation information. For example, the disinfestation displaying module 210 may display disinfestation information on a screen of the disinfestation estimating device 210. Additionally or alternatively, the disinfestation displaying module 210 may display disinfestation information on a screen of another device. For example, the disinfestation displaying module 210 may display disinfestation information on a screen of another device that may be communicatively linked with the disinfestation estimating device 200, such as a mobile communication device. Furthermore, in some examples, responsive to estimating the disinfestation rate, parameters for the disinfestation equipment may be adjusted. For example, any one or combination of the parameters for the disinfestation equipment discussed above may be adjusted, and these adjustments to the parameters may be made in any one or combination of the manners discussed above.

Turning to FIGS. 3-4, example methods are shown. In at least one example, the methods described in FIGS. 3-4 may be stored as instructions executable by the logic subsystem of the computing system as described at FIG. 2 to implement the herein described methods.

Turning now to FIG. 3, a flow chart of an example method 300 for estimating a disinfestation rate is shown. Method 300 may begin at 301, where infestation information may be received. In one example, this information may be received by the infestation information receiving module as described at FIG. 2. For example, the infestation information at step 301 may be received via a user input in any one or combination of the manners described above.

Responsive to receiving infestation information, as step 302 method 300 may include determining a number of products that are infested in an untreated sample out of a total number of products in the untreated sample based on the received infestation information. The number of products that are infested in the untreated sample out of the total number of products in the treated sample may be determined based on the received infestation information.

At step 304 of method 300, method 300 may include determining a number of infested products in a treated sample out of a total number of products in the treated sample based on the received infestation information. Once infestation information has been determined for both an untreated sample of products and a treated sample of products, a sampling event may be complete.

In some examples, a sampling event may take place for each treatment, which may be advantageous for gathering a larger number of discrete samples, that is, a larger number of products that are sampled. Additionally, having a sampling event for each treatment may be advantageous for quickly detecting if there are any issues with achieving a desired disinfestation rate that may require intervening action, such as making adjustments to the disinfestation equipment. However, in some examples a sampling event may take place intermittently or at regular intervals that have treatments in between sampling.

Once the infestation information has been determined at step 302 and step 304 of method 300, method 300 may include estimating the disinfestation rate. The disinfestation rate may be estimated based on one or both of the information determined at step 302 and the information determined at step 304, for example. Additionally, the disinfestation rate may be estimated based on historical infestation information, so that the disinfestation rate may be an ongoing estimated disinfestation rate. This historical infestation information may be based on previous sampling events, for example. More details regarding calculations and models for estimating the disinfestation rate are discussed in relation to FIG. 4.

Following estimating the disinfestation rate at step 306, method 300 may include generating a disinfestation display of the estimated disinfestation rate determined at step 308. Additionally or alternatively, the disinfestation display may be generated based on calculations and models used to determine the estimated disinfestation rate. For example, while estimating the disinfestation rate, various calculations may be performed and various models may be generated while estimating the disinfestation rate, and any one or combination of these calculations and models may be displayed. Furthermore, estimating the disinfestation rate may include performing a pass-fail calculation, and the results of this pass-fail calculation may additionally or alternatively be displayed. For example, if the estimated disinfestation rate fails based on the comparison to the pass-fail curve, it may be determined that the estimated disinfestation rate is less than a desired disinfestation rate. If the estimated disinfestation rate may be determined to pass, it may be determined that estimated disinfestation rate is greater than or equal to the desired disinfestation rate. In at least one example, the desired disinfestation rate may be based on a disinfestation rate to meet food quality standards. Generating the disinfestation display may include either updating a display already being displayed or generating a new display.

Following generating the disinfestation display, step 310 of method 300 a disinfestation display may include displaying generated disinfestation display. For example, the display may be provided on a screen of a disinfestation estimating device. Additionally or alternatively, displaying a disinfestation display based on the estimated disinfestation rate may include providing a display on a screen of a device communicatively linked to the disinfestation estimating device.

At step 312 of method 300, operating parameters of the disinfestation equipment may be adjusted. For example, any one or combination of the operating parameters described above may be adjusted. In some examples, the operating parameters of the disinfestation equipment may be adjusted responsive to the estimated disinfestation rate being less than a desired disinfestation rate. In examples where the operating parameters of the disinfestation equipment may be adjusted responsive to the estimated disinfestation equipment being less than the desired disinfestation rate, the operating parameters of the disinfestation equipment may be adjusted to create conditions that are harsher in regards to the organism that is to be eradicated. For example, a temperature may be further increased, a processing time may be increased, a temperature may be further decreased, and an amount of a chemical applied may be increased. However, any operating parameters may be adjusted that may increase the estimated disinfestation rate of the equipment to be greater than or equal to the desired disinfestation rate.

Furthermore, in some examples, operating parameters of the disinfestation equipment may be adjusted in response to the estimated disinfestation rate being greater than or equal to the desired disinfestation rate. For example, operating parameters of the disinfestation equipment may be adjusted in order to optimize energy use of the disinfestation equipment to achieve the desired disinfestation rate while not over expending energy.

As discussed above, adjustments to the operating parameters of the disinfestation equipment may be carried out automatically or manually.

Following step 312 of method 300, in at least one example method 300 may include disposing of the treated sample and the untreated sample at step 314. As discussed above, during inspection of food products that are selected for the untreated and treated samples, the food products may be rendered unfit for sale. For example, during inspection, the food products may need to be cut open to inspect the food products for infestation, and this cutting open of the food products may render the food products unfit for sale, in at least one example. In such examples where the untreated sample and the treated sample are rendered unfit for sale during inspection, method 300 may include disposing these products. It is noted that while step 314 is shown taking place after adjusting the parameters of the equipment, step 314 may take place at any time after determining infestation information for the untreated sample and the treated sample, at steps 302 and 304, respectively. For example, the untreated sample may be disposed of any time after step 302 of method 300 and the treated sample may be disposed of any time after step 304 of method 300. Furthermore, in examples where determining the information at steps 302 or 304 may not be destructive, in other words, not render the food products unfit for sale, method 300 may not include step 314.

Turning now to FIG. 4, a flow chart of an example method 400 for calculating a disinfestation rate estimate is shown. In at least one example, method 400 may correspond to step 306 of method 300. Method 400 may begin at step 402 by estimating a survival rate. In at least one example, the survival rate may be estimated based on infestation information for a single sampling event. As discussed above, a sampling event may be an event in which infestation information for an untreated sample and a treated sample is determined.

In one example, a first step for estimating the survival rate for the single sampling event may be as follows:

Z=Θ/Λ

Where Λ and Θ are defined as random variables, with Λ=mean infestation rate (e.g. mean live insects per product) before treatment and with Θ=mean infestation rate after treatment. In some examples the product may be a food product such as a date. The mean infestation rate and the mean infestation rate after treatment random variables may take into account an underlying probability distribution for arrival events of insects due to eggs laid by the insects. The underlying probability distribution for arrival events may be modeled using a Poisson distribution, and a number of insects in each cluster may be assumed to follow a known distribution. For example, the number of insects that may hatch from each cluster may be assumed to follow a uniform distribution. Alternatively, other distributions may be used based on a particular insect that may be found within the products to more closely model the arrival of the clusters and a number of insects that may hatch from each cluster.

Furthermore, when estimating the survival rate for a sampling event, a survival likelihood curve function for Z (where Z may be defined as described above) may then be determined. In one example the survival likelihood curve, f_{z}(z), in other words, the likelihood function of Z, may be calculated as a probability of functions for the infestation rate for the infestation rate before and after a treatment. For example, a survival likelihood curve for a single sampling event, f_{z}(z), may be calculated as follows: The quotient of two random variables may be defined as Z=Θ/Λ.

That is, given f_{Θ}(θ) and f_{Λ}(λ)→find f_{z}(z).

$$\begin{matrix}{F}_{Z} ( z ) = P ( Z \leq z ) \\ = P ( \frac{Θ}{Λ} \leq z ) \\ = P ( ( \frac{Θ}{Λ} \leq z ) ⋂ ( ( Λ \geq 0 ) ⋃ ( Λ < 0 ) ) \\ = P ( \frac{Θ}{Λ} \leq z , Λ \geq 0 ) + P ( \frac{Θ}{Λ} \leq z , Λ < 0 ) \\ = P ( Θ \leq Λ z , Λ \geq 0 ) + P ( Θ \leq Λ z , Λ < 0 )\end{matrix}$$

$${F}_{z} ( z ) = {\int }_{λ = 0}^{∞} {\int }_{θ = - ∞}^{Λ z} {f}_{ΘΛ} ( θ , λ ) d θ d λ + {\int }_{λ = - ∞}^{0} {\int }_{θ = λ z}^{∞} {f}_{ΘΛ} ( θ , λ ) d θ d λ$$

Apply Leibniz's integral rule,

$$\begin{matrix}{Leibniz}^{′} s integral rule \text{:} \\ \frac{d}{dt} {\int }_{a ( t )}^{b ( t )} f ( x , t ) dx = {\int }_{a ( t )}^{b ( t )} \frac{\partial f}{\partial t} dx + f ( b ( t ) , t ) \cdot {b}^{′} ( t ) - f ( a ( t ) , t ) \cdot {a}^{′} ( t )\end{matrix}$$

$${f}_{z} ( z ) = \frac{d}{dz} {F}_{z} ( z ) \text{:}$$

$${f}_{z} ( z ) = {\int }_{λ = 0}^{∞} λ \cdot {f}_{ΘΛ} ( λ z , λ ) d λ + {\int }_{λ = - ∞}^{0} - λ \cdot {f}_{ΘΛ} ( λ z , λ ) d λ = {\int }_{λ = 0}^{∞} \vert λ \vert \cdot {f}_{ΘΛ} ( λ z , λ ) d λ$$

If and only if Λ and Θ are independent, f_{ΘΛ}(λz, λ)=f_{Θ}(λz)·f_{Λ}(λ)

$${ }^{Λ} {f}_{z} ( z ) = {\int }_{- ∞}^{∞} \vert λ \vert \cdot {f}_{Θ} ( λ z ) \cdot {f}_{Λ} ( λ ) d λ = {\int }_{0}^{∞} λ \cdot {f}_{Θ} ( λ z ) \cdot {f}_{Λ} ( λ ) d λ$$

$$if λ \geq 0$$

Based on f_{z}(z), a most likely survival rate for the single sampling event may be estimated. Additionally or alternatively, a disinfestation rate for this single sampling event may be estimated by subtracting the most likely survival rate from 1.

Following estimating a survival rate at step 402, step 404 of method 400 may include updating an aggregated likelihood function of the survival rate with the survival likelihood curve for the single sampling event calculated at step 402.

The aggregate likelihood function of the survival rate may take into account multiple sampling events and may be generated by performing a conflation operation to incorporate the information from each new sample event into the updated aggregated estimate. In one example, the aggregated likelihood function may be generated by combining multiple survival likelihood curves for single sampling events. For example, the aggregated likelihood function may be updated via conflation. The conflation may be described as below:

Where conflation of f₁(x), f₂(x), . . . fₙ(x) is defined as f(x) below, and where each of f₁(x), f₂(x), . . . fₙ(x) are survival likelihood curves for individual sampling events as described above:

$$f ( x ) = \frac{{f}_{1} ( x ) {f}_{2} ( x ) … {f}_{n} ( x )}{{\int }_{- ∞}^{∞} {f}_{1} ( t ) {f}_{2} ( t ) … {f}_{n} ( t ) dt}$$

And a stepwise conflation calculation derivation may be defined as below:

For z: z₁→zₘ

$${f}_{c 1} ( z ) = \frac{{f}_{1} ( z )}{{f}_{1} ( {z}_{1} ) + {f}_{1} ( {z}_{2} ) + {f}_{1} ( {z}_{3} ) + … + {f}_{1} ( {z}_{m} )}$$

$${f}_{c 2} ( z ) = \frac{{f}_{1} ( z ) \cdot {f}_{2} ( z )}{{f}_{1} ( {z}_{1} ) \cdot {f}_{2} ( {z}_{1} ) + {f}_{1} ( {z}_{2} ) \cdot {f}_{2} ( {z}_{2} ) + … + {f}_{1} ( {z}_{m} ) \cdot {f}_{2} ( {z}_{m} )}$$

$${f}_{c 2} ( z ) = {f}_{c 1} ( z ) \cdot \frac{{f}_{1} ( {z}_{1} ) + {f}_{1} ( {z}_{2} ) + … + {f}_{1} ( {z}_{m} )}{{f}_{1} ( {z}_{1} ) \cdot {f}_{2} ( {z}_{1} ) + {f}_{1} ( {z}_{2} ) \cdot {f}_{2} ( {z}_{2} ) + … + {f}_{1} ( {z}_{m} ) \cdot {f}_{2} ( {z}_{m} )} \cdot {f}_{2} ( z )$$

Define f₁^{˜}(zᵢ) as:

$${f}_{1}^{∼} ( {z}_{i} ) = \frac{{f}_{1} ( {z}_{i} )}{{f}_{c 1} ( {z}_{i} )} = \frac{{f}_{1} ( {z}_{i} )}{{f}_{1} ( {z}_{1} ) + {f}_{1} ( {z}_{2} ) + … + {f}_{1} ( {z}_{m} )}$$

$$\begin{matrix}{f}_{c 2} ( z ) = {f}_{c 1} ( z ) \cdot \frac{1}{\begin{matrix}{f}_{1}^{∼} ( {z}_{1} ) \cdot {f}_{2} ( {z}_{1} ) + {f}_{1}^{∼} ( {z}_{2} ) \cdot \\ {f}_{2} ( {z}_{2} ) + … + {f}_{1}^{∼} ( {z}_{m} ) \cdot {f}_{2} ( {z}_{m} )\end{matrix}} \cdot {f}_{2} ( z ) \\ = \frac{{f}_{c 1} ( z ) \cdot {f}_{2} ( z )}{{f}_{1}^{∼} ( {z}_{1} ) \cdot {f}_{2} ( {z}_{1} ) + {f}_{1}^{∼} ( {z}_{2} ) \cdot {f}_{2} ( {z}_{2} ) + … + {f}_{1}^{∼} ( {z}_{m} ) \cdot {f}_{2} ( {z}_{m} )}\end{matrix}$$

$${f}_{c 3} ( z ) = \frac{{f}_{1} ( z ) \cdot {f}_{2} ( z ) \cdot {f}_{3} ( z )}{\begin{matrix}{f}_{1} ( {z}_{1} ) \cdot {f}_{2} ( {z}_{1} ) \cdot {f}_{3} ( {z}_{1} ) + \\ {f}_{1} ( {z}_{2} ) \cdot {f}_{2} ( {z}_{2} ) \cdot {f}_{3} ( {z}_{2} ) + … + {f}_{1} ( {z}_{m} ) \cdot {f}_{2} ( {z}_{m} ) \cdot {f}_{3} ( {z}_{m} )\end{matrix}}$$

$${f}_{c 3} ( z ) = \frac{{f}_{1} ( {z}_{1} ) \cdot {f}_{2} ( {z}_{1} ) + {f}_{1} ( {z}_{2} ) \cdot {f}_{2} ( {z}_{2} ) + … + {f}_{1} ( {z}_{m} ) \cdot {f}_{2} ( {z}_{m} )}{\begin{matrix}{f}_{1} ( {z}_{1} ) \cdot {f}_{2} ( {z}_{1} ) \cdot {f}_{3} ( {z}_{1} ) + \\ {f}_{1} ( {z}_{2} ) \cdot {f}_{2} ( {z}_{2} ) \cdot {f}_{3} ( {z}_{2} ) + … + {f}_{1} ( {z}_{m} ) \cdot {f}_{2} ( {z}_{m} ) \cdot {f}_{3} ( {z}_{m} ) \cdot {f}_{3} ( z )\end{matrix}}$$

Define f₂^{˜}(zᵢ) as:

$${f}_{2}^{∼} ( {z}_{i} ) = \frac{{f}_{1} ( {z}_{i} )}{{f}_{c 2} ( {z}_{i} )} = \frac{{f}_{1} ( {z}_{i} ) \cdot {f}_{2} ( {z}_{i} )}{{f}_{1} ( {z}_{1} ) \cdot {f}_{2} ( {z}_{1} ) + {f}_{1} ( {z}_{2} ) \cdot {f}_{2} ( {z}_{2} ) + … + {f}_{1} ( {z}_{m} ) \cdot {f}_{2} ( {z}_{m} )}$$

$$\begin{matrix}{f}_{c 3} ( z ) = {f}_{c 2} ( z ) \cdot \frac{1}{\begin{matrix}{f}_{2}^{∼} ( {z}_{1} ) \cdot {f}_{3} ( {z}_{1} ) + {f}_{2}^{∼} ( {z}_{2} ) \cdot \\ {f}_{3} ( {z}_{2} ) + … + {f}_{2}^{∼} ( {z}_{m} ) \cdot {f}_{3} ( {z}_{m} )\end{matrix}} \cdot {f}_{3} ( z ) \\ = \frac{{f}_{c 2} ( z ) \cdot {f}_{3} ( z )}{{f}_{c 2} ( {z}_{1} ) \cdot {f}_{3} ( {z}_{1} ) + {f}_{c 2} ( {z}_{2} ) \cdot {f}_{3} ( {z}_{2} ) + … + {f}_{c 2} ( {z}_{m} ) \cdot {f}_{3} ( {z}_{m} )}\end{matrix}$$

$${}_{}^{Λ}{f}{}_{c , n}^{} ( z ) = \frac{{f}_{c , n - 1} ( z ) \cdot {f}_{n} ( z )}{{f}_{c , n - 1} ( {z}_{1} ) \cdot {f}_{n} ( {z}_{1} ) + {f}_{c , n - 1} ( {z}_{2} ) \cdot {f}_{n} ( {z}_{2} ) + … + {f}_{c , n - 1} ( {z}_{m} ) \cdot {f}_{n} ( {z}_{m} )}$$

By generating this survival likelihood curve which combines the estimated survival rate across multiple sampling events, an accuracy of the estimated disinfestation rate may be improved and an uncertainty for the estimated disinfestation rate may be reduced.

After updating the aggregated likelihood function of the survival rate at step 404, method 400 may include estimating the disinfestation rate based on the updated aggregated likelihood function of the survival rate at step 406. For example, based on the updated aggregated likelihood function of the survival rate from 404, a maximum likelihood estimate may be determined, where the maximum likelihood estimate is an estimate of the survival rate based on the updated aggregated likelihood function of the survival rate that is most likely to be the actual survival rate.

The maximum likelihood estimate, also referred to as the zMLE, may be determined by analyzing the updated aggregated likelihood curve. For example, the zMLE may be determined based on a z-value at a peak of the plotted updated aggregate likelihood curve, where the z-value is a survival ratio value positioned on an x-axis of the updated aggregated likelihood function of the survival rate. The disinfestation rate may then be defined as 1 minus the survival rate, where the survival rate may be based on the maximum likelihood estimate

For example, if it is determined based on the updated aggregate likelihood function of the survival rate at 404 that there is a maximum likelihood survival rate of 0.001, then the disinfestation rate would be 1-0.001, equating to a disinfestation rate of 0.999, or 99.9%. This disinfestation estimate may be determined based on discrete samples taken before and after treatments. For example, in some embodiments samples may be taken before and after every treatment. However, in other examples, samples may not be taken before and after every treatment, and the samples may instead be taken intermittently or at regular intervals.

After estimating the disinfestation rate based on the aggregated likelihood function of the survival rate at step 406, method 400 may include comparing the estimated disinfestation rate to a pass-fail curve at step 408. In some examples, the pass-fail curve may be generated as is described at FIG. 6.

Comparing the estimated disinfestation rate to the pass-fail curve may include plotting the maximum likelihood estimate for a survival rate, which is determined based on the updated aggregated likelihood function of the survival rate, where the aggregated likelihood function of the survival rate may be updated as described at step 406.

For example, after each sampling event, a maximum likelihood estimate for the survival rate of that sampling event may be determined based on the updated aggregated likelihood function of the survival rate. In some examples, the maximum likelihood estimate for the survival rate may be determined as is described in relation to step 408. After determining the maximum likelihood estimate for the survival rate of the sampling event, this maximum likelihood estimate may be plotted on a same plot as the pass-fail curve.

The process of determining the maximum likelihood estimate of the survival rate for a sampling event and plotting the determined maximum likelihood estimate of the survival rate on the same plot as a pass-fail plot may be repeated each sampling event to generate an ongoing estimate of the survival rate. Then, based on where the plot of the maximum likelihood estimates of the survival rate (i.e., the ongoing estimate) converges relative to the pass-fail curve, an estimated disinfestation rate may be determined to pass or fail. For example, if the maximum likelihood estimates for each of the sampling events converges in a region of the pass-fail curve that is pre-determined to be a pass region, then the estimated disinfestation rate is indicated to pass. Additionally, if the estimated disinfestation rate converges in a region of the pass-fail curve that is pre-determined to be a fail region of the curve, then the estimated disinfestation rate is indicated to fail. In at least one example, the ongoing estimate must be in either a pass region or a fail region of the pass-fail curve for greater than a threshold number of samples before concluding that the ongoing estimate passes or fails. If the ongoing estimate is determined to fail, then it is determined that the disinfestation rate is less than a desired disinfestation rate. If the ongoing estimate is determined to pass, then the estimated disinfestation rate is determined to be greater than or equal to the desired disinfestation rate.

In at least one example, after the estimated disinfestation rate passes or fails based on the comparison to the pass-fail curve, then the disinfestation estimating device may provide an indication that the disinfestation equipment has an estimated disinfestation rate that passes or fails.

Providing an indication that the disinfestation equipment has a passing or a failing estimated disinfestation rate may include any one or combination of displaying an alert on a screen of the disinfestation estimating device and displaying an alert on a screen of a device communicatively linked with the disinfestation estimating device. Additionally or alternatively, providing an indication that the disinfestation equipment has a passing or a failing estimated disinfestation rate may include generating an audio indication, such as a ringing or beeping sound.

Turning now to FIG. 5, FIG. 5 shows a graph of an example aggregated likelihood function for a survival rate 500. The graph of the aggregated likelihood function of the survival rate 500 may include survival ratio values (z) across an x-axis of the aggregated likelihood function. The y-axis of the graph of the aggregated likelihood function of the survival rate may be values for the aggregated likelihood function of the survival rate indicating a likelihood that the values on the x-axis are the actual survival rate. For example, the greater the y-value, the greater the likelihood that the survival ratio values on the x-axis that corresponds with that y-value is the actual survival ratio. In some examples, the aggregated likelihood function of the survival rate may be curves of each historical survival likelihood curves, f_{z}(z), that have been combined into the aggregated likelihood function of the survival rate, £(z), through conflation of the f_{z}(z) curves, as described above in reference to FIG. 4.

The resulting curve 506 may thus be an aggregated likelihood function of the survival rate, and a peak 502 of the aggregated likelihood function of the survival rate may correspond to a maximum likelihood estimate for the survival rate. In particular, a z-value 504 at a peak 502 of the aggregated likelihood function of the survival rate may be a maximum likelihood estimate for the survival rate (zMLE). Put another way, the peak 502 of the aggregated likelihood function curve 506 may correspond to a zMLE 504, where the zMLE is a survival ratio 504 that is most likely to be the actual survival ratio. In some examples, this zMLE may be used to estimate a disinfestation rate. For example, in the graph shown at FIG. 5, a survival ratio of approximately 0.0005 appears to be the zMLE. Thus, the disinfestation rate may be estimated to be 1-0.0005, for a 0.9995 disinfestation rate or a 99.95% disinfestation rate.

Turning now to FIG. 6, FIG. 6 shows a graph of an example pass-fail curve plotted against survival rate estimate 600. An x-axis of the graph may be a number of samples tested. The y-axis may be a survival rate estimate.

In at least one example, the pass-fail curve plotted against the survival rate estimate 600 may be filtered to determine a survival rate for an infestation in a certain stage. For example, the graph in FIG. 6 has been filtered to plot the pass-fail curve 602 against an estimated survival rate 604 for an infestation that is in a larvae stage. Additionally or alternatively, filters may be applied to determine the survival rate for an infestation in one or both of an adult stage and an egg stage.

In some examples, this filtering process to determine the survival rate for an infestation in a certain stage may separate the stages of the infestation by applying a distribution to account for the stages the estimated survival rate 604 and to the pass-fail curve 602. In other examples, however, the stage of the infestation may be input at the time of determining the infestation information for the treated and untreated samples in a sampling event, and the pass-fail curve 602 and the estimated survival rate 604 may then only be plotted based upon infestation information that was tagged as being for a certain stage of an infestation, such as an egg, larvae, or adult stage of an infestation.

The pass-fail curve 602 may be generated based on Monte Carlo simulations which are performed with the known underlying disinfestation rates based on the determined infestation information to calculate an uncertainty of the survival rate estimates. These Monte Carlo simulations may be used to generate 99^{th} percentile curves based on sampling parameters (e.g., infestation information) to generate the pass-fail curve 602, and these Monte Carlo simulations may be performed each sampling event in order to update the 99^{th} percentile pass-fail curve 602.

The pass-fail curve 602 may separate the graph into two regions: a pre-determined fail region 606 and a pre-determined pass region 608. The survival rate estimate 604 may then be plotted against the pass-fail curve 602. In at least one example, the survival rate estimate 604 may be zMLE values that are plotted following each sampling event. Thus, the survival rate estimate 604 may be based on estimates that are already processed to help ensure that this survival rate estimate 604 is accurate. As this survival rate estimate 604 is plotted over multiple sampling events, the survival rate estimate 604 is an ongoing estimate of the survival rate.

After the survival rate estimate 604 has been plotted against the pass-fail curve 602, the survival rate estimate 604 may be determined to pass or fail based on whether the survival rate estimate 604 converges in the pre-determined pass region 608 or the pre-determined fail region 606. For example, if the survival rate estimate 604 converges in the pre-determined pass region 608, then it may be determined that the disinfestation rate is at least a desired disinfestation rate. As discussed above, the disinfestation rate is directly related to the survival rate, as the disinfestation rate is 1 minus the estimated survival rate. On the other hand, if the survival rate estimate 604 converges in the pre-determined fail region 606, then it may be determined that the disinfestation rate may not be at least the desired disinfestation rate. In examples where the survival rate estimate 604 may converge at the pass-fail curve 602, it may be determined that more samples are needed to conclude whether or not the disinfestation rate passes or fails. Alternatively, in at least one example, the survival rate estimate 604 may yield a fail if the disinfestation rate estimate 604 converges at the pass-fail curve.

Once the survival rate estimate 604 is determined to either pass or fail, this information may be displayed. For example, a disinfestation estimating module may estimate the disinfestation rate and determine whether the estimated survival rate passes or fails, and then the disinfestation estimating device may display the disinfestation information. For example, the disinfestation estimating device may display any one or combination of an estimated disinfestation rate, an indication of whether the estimated survival rate passes or fails when compared to the pass-fail curve, a graph of the pass-fail curve 602 plotted against the estimated survival rate 604, a graph of the aggregated likelihood function for the survival rate 500, and infestation information in a form of a spreadsheet.

Thus, provided are methods for estimating a disinfestation rate of disinfestation equipment to achieve a desired disinfestation rate. In a first example of the method, a disinfestation rate may be estimated based on a number of infested products in an untreated sample out of a total number of products in the untreated sample and based on a number of infested products in a treated sample out of a total number of products in the treated sample, and the estimated disinfestation rate may be displayed. A second example which optionally includes the first example, may include wherein the products in the untreated sample have not been treated by disinfestation equipment, and wherein the products in the treated sample have been treated by the disinfestation equipment. Additionally, an example of the method which may optionally include one or both the first and second examples of the method, may further include wherein the infested products are products that contain any live infestation. In a further example of the method, which may include any one or combination of the above example methods, estimating the disinfestation rate may include estimating a survival rate based on the number of infested products out of the total number of products for both the untreated sample and the treated sample, and updating an aggregated likelihood function of the survival rate with the estimated survival rate. A still further example of the method which may optionally include any one or combination of the above described examples may further comprise displaying calculations used to estimate the disinfestation rate. In at least one example, the calculations used to estimate the disinfestation rate may include calculations to determine an ongoing estimate of a survival rate.

In still another example method that may optionally include any one or combination of the above examples, estimating a disinfestation rate of disinfestation equipment may include receiving infestation information, estimating a disinfestation rate based on the received infestation information, and adjusting parameters of disinfestation equipment in response to the estimated disinfestation rate. Additionally, in a method which may optionally include any one or combination of the above examples, estimating the disinfestation rate may include comparing an ongoing estimate of a survival rate with a pass-fail curve. In at least one example, the ongoing estimate of the survival rate may be based upon the received infestation information and historical infestation information. Additionally or alternatively, the infestation information received may be infestation information for a sample of treated products and infestation information for a sample of untreated products. In an example of the method, which may optionally include any one or a combination of the above example methods described, adjusting the parameters of the disinfestation equipment may include any one or combination of adjusting a processing time for a treatment performed by the disinfestation equipment and adjusting a temperature threshold. Additionally, in at least one example the parameters of the disinfestation equipment may be adjusted in response to the estimated disinfestation rate being less than a desired disinfestation rate.

Further still, another example of the method which may optionally include any one or combination of the above described examples may include treating a portion of food products out of a total number of food products with disinfestation equipment, and leaving a remainder of the total number of food products untreated by the disinfestation equipment, determining infestation information for the food products untreated by the disinfestation equipment, determining infestation information for the food products treated with the disinfestation equipment, and estimating a disinfestation rate of the disinfestation equipment based on the determined infestation information for the food products untreated by the disinfestation equipment and the food products treated with the disinfestation equipment. In some examples, the method may include displaying the estimated disinfestation rate of the disinfestation equipment. Additionally, in another example of the method which may optionally include any one or combination of the above described methods, the operating parameters of the disinfestation equipment may be adjusted responsive to the estimated disinfestation rate of the disinfestation equipment. For example, the operating parameters of the disinfestation equipment may be adjusted responsive to the estimated disinfestation rate of the disinfestation equipment being less than a desired disinfestation rate. Regarding estimating the disinfestation rate, in an example method which may optionally include any one or combination of the above methods, estimating the disinfestation rate may include estimating a survival rate based on the infestation information determined for the untreated food products and the infestation information determined for the treated food products. Additionally or alternatively, estimating the disinfestation rate may further include updating an aggregated likelihood function of the survival rate with the estimated survival rate. Furthermore, in some examples estimating the disinfestation rate may further include estimating the disinfestation rate based on a maximum likelihood estimate from the updated aggregated likelihood function of the survival rate.

It will be appreciated that the configurations and/or approaches described herein are exemplary in nature, and that these specific embodiments or examples are not to be considered in a limiting sense, because numerous variations are possible. The subject matter of the present disclosure includes all novel and nonobvious combinations and subcombinations of the various features, functions, acts, and/or properties disclosed herein, as well as any and all equivalents thereof.

1. A method, comprising:
estimating a disinfestation rate based on a number of infested products in an untreated sample out of a total number of products in the untreated sample and based on a number of infested products in a treated sample out of a total number of products in the treated sample; and
displaying the estimated disinfestation rate.

2. The method of claim 1, wherein the products in the untreated sample have not been treated by disinfestation equipment, and wherein the products in the treated sample have been treated by the disinfestation equipment.

3. The method of claim 1, wherein the infested products are products that contain any live infestation.

4. The method of claim 1, wherein estimating the disinfestation rate includes estimating a survival rate based on the number of infested products out of the total number of products for both the untreated sample and the treated sample, and updating an aggregated likelihood function of the survival rate with the estimated survival rate.

5. The method of claim 1, further comprising displaying calculations used to estimate the disinfestation rate.

6. The method of claim 4, wherein the calculations used to estimate the disinfestation rate include calculations to determine an ongoing estimate of a survival rate.

7. A method, comprising:
receiving infestation information;
estimating a disinfestation rate based on the received infestation information; and
adjusting parameters of disinfestation equipment in response to the estimated disinfestation rate.

8. The method of claim 7, wherein estimating the disinfestation rate includes comparing an ongoing estimate of a survival rate with a pass-fail curve.

9. The method of claim 8, wherein the ongoing estimate of the survival rate is based upon the received infestation information and historical infestation information.

10. The method of claim 7, wherein the infestation information received is infestation information for a sample of treated products and infestation information for a sample of untreated products.

11. The method of claim 7, wherein adjusting the parameters of the disinfestation equipment includes adjusting a processing time for a treatment performed by the disinfestation equipment.

12. The method of claim 7, wherein adjusting the parameters of the disinfestation equipment includes adjusting a temperature threshold.

13. The method of claim 7, wherein the parameters of the disinfestation equipment are adjusted in response to the estimated disinfestation rate being less than a desired disinfestation rate.

14. A method, comprising:
treating a portion of food products out of a total number of food products with disinfestation equipment, where a remainder of the total number of food products are untreated by the disinfestation equipment;
determining infestation information for the food products treated with the disinfestation equipment;
determining infestation information for the food products untreated by the disinfestation equipment; and
estimating a disinfestation rate of the disinfestation equipment based on the determined infestation information for the food products untreated by the disinfestation equipment and the food products treated with the disinfestation equipment.

15. The method of claim 14, further comprising displaying the estimated disinfestation rate of the disinfestation equipment.

16. The method of claim 14, further comprising adjusting operating parameters of the disinfestation equipment responsive to the estimated disinfestation rate of the disinfestation equipment.

17. The method of claim 16, wherein the operating parameters of the disinfestation equipment are adjusted responsive to the estimated disinfestation rate of the disinfestation equipment being less than a desired disinfestation rate.

18. The method of claim 14, wherein estimating the disinfestation rate includes estimating a survival rate based on the infestation information determined for the untreated food products and the infestation information determined for the treated food products.

19. The method of claim 18, wherein estimating the disinfestation rate further includes updating an aggregated likelihood function of the survival rate with the estimated survival rate.

20. The method of claim 19, wherein estimating the disinfestation rate further includes estimating the disinfestation rate based on a maximum likelihood estimate from the updated aggregated likelihood function of the survival rate.
